(19) United States
(12) Patent Application Publication
(10) Pub. No.: US 2019/0076057 A1
(43) Pub. Date: Mar. 14, 2019

(54) FILTER

(71) Applicant: ORIDION MEDICAL 1987 LTD., Jerusalem (IL)

(72) Inventors: Michael Kertser, Bney Aish (IL); Konstantin Goulitski, Holon (IL)

(21) Appl. No.: 16/123,086

(22) Filed: Sep. 6, 2018

Related U.S. Application Data
(60) Provisional application No. 62/558,096, filed on Sep. 13, 2017.

(51) Int. Cl. A61B 5/097; A61B 5/08; A61M 16/10; A61M 16/08

(52) U.S. Cl. CPC A61B 5/097; A61B 5/082; A61M 2230/432; A61M 16/085; A61M 16/1065

(57) ABSTRACT

A fluid filtering device for separating liquid from gases includes a filter located within the lumen of the filtering device. The filter blocks materials other than gases from passing therethrough. A element fluidly coupled to the filter provides an indication to a user once liquid in the lumen is at a predetermined threshold level.

### CROSS-REFERENCE TO RELATED APPLICATION

This application claims priority and the benefit of U.S. Provisional Application Ser. No. 62/558,096 entitled “FILTER,” filed Sep. 13, 2017, the content of which is incorporated herein by reference in its entirety for all purposes.

### TECHNICAL FIELD

The present disclosure generally relates to a fluid filtering device, specifically to a fluid filtering device including a mechanism for preventing reuse thereof.

### BACKGROUND

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Breath sampling tubes with inline filters used in combination with CO₂ monitoring systems (e.g., a capnograph) generally retain a predefined amount of liquids (e.g., approximately 135 μL). Once an absorption and/or accumulation limit of the liquid within the inline filter has been reached, the CO₂ monitor will detect a pressure drop. In response to the detected pressure drop, the monitoring system may cease working to mitigate damage that may be caused by undesirable fluids entering the breath monitoring system, and the monitoring system may output an alert to medical staff instructing them to change the consumable (e.g., the inline filter). However, the inline filters may be reused after cleaning (e.g., with compressed air). Reuse of the inline filter may be undesirable due, in part, to health concerns associated with used filters. Accordingly, it is now recognized that there is a need for an inline filter that includes features to prevent its reuse once the filter has meet the predetermined absorption and/or accumulation limit.

### SUMMARY

The present disclosure relates to fluid filtering devices (e.g., filter-containing disposable), including a mechanism that may prevent reuse thereof.

According to some embodiments, the filtering device disclosed herein may include an element that may provide an indication to a user once the liquid in the filter reaches a predetermined threshold level and/or an indication as to the level of liquids absorbed and/or accumulated within the filter. According to some embodiments, the indication may be an irreversible visual indication, such as, but not limited to, a color indication. That is, once the liquid having entered the filter reaches the predetermined threshold level, the filter and/or an indicator incorporated therein, changes its property in such way that both the medical staff as well as the patient are informed of the filter having reached its maximum capacity, even if dried and/or cleaned. In this way, the filter cannot be reused without the patient or their caregivers being aware that a used filter is connected to the monitoring system.

According to some embodiments, the element may include features that block reuse of the filter. For example, the filter may include a mechanism that may cease operation of a medical device (e.g., capnograph) to which it is connected, or otherwise interfere with the reuse of the filter. This may actively prevent reuse of the filter. The filter may include an element fluidly connected to the filter, the element may, for example, include an electric circuit that may close or open when the liquid in the filter reaches a predetermined threshold level. By way of non-limiting example, the element may include a conductive substance that may lose its conductivity when exposed to contact with liquids. In certain embodiments, the element may include a substance that may close the electric circuit as a result of being exposed to liquids. The element may include low cost components to decrease manufacturing costs of the filter.

According to some embodiments, there is provided a fluid filtering device that may be used to separate liquid from gases (e.g., CO₂) to be analyzed by the CO₂ monitoring system. The fluid filtering device includes a wall forming a lumen having an inlet at a proximal end thereof, an outlet at a distal end thereof, and a filter located within the lumen between the proximal and distal ends. The filter may block undesirable components other than gases to pass therethrough and into a patient's airway. The fluid filtering device may also include an element fluidly connected to the filter. For example, the element may be positioned within the wall of the lumen. The element may provide an indication to a user (e.g., a patient and/or a caregiver) that the liquid within the lumen is at or has previously reached a predetermined threshold level. For example, in certain embodiments, the element may change a visual property of the fluid filtering device when the liquid within the lumen is at the predetermined threshold level. In some embodiments, the element may include an electric circuit that closes/opens when the liquid in the lumen is at the predetermined threshold level.

According to some embodiments, the element may include a substance that may irreversibly change its property due, in part, to interaction with the liquid. In certain embodiments, the substance may react with the liquid. For example, the substance may be a conductive substance. The liquid within the fluid filtering device may react with the substance such that the electric circuit of the element closes. According to some embodiments, the substance may swell upon interaction with the liquid. Swelling of the substance within the fluid filtering device may close the electrical circuit. In certain embodiments, the element may include or be a capillary channel that may receive fluids when the liquid in the lumen reaches the predetermined threshold level. The substance may be contained within the capillary channel. By way of non-limiting example, the substance may be or include sodium polyacrylate.

According to some embodiments, there is provided a breath sampling tube including a filter section having a filter located within a lumen of the filter section. The filter section may be integral to the breath sampling tube. However, in certain embodiments, the filter section may be a separate element connectable to the breath sampling tube. The filter in the filter section may block components other than gases from passing therethrough; and an element fluidly connected to the filter provides an indication to a user that liquid accumulated in the lumen is at or has previously reached a predetermined threshold level. The element may change a visual property of at least a portion of the breathing sample tube (e.g., the filter section, the filter, and/or the element), when the liquid accumulated in the lumen reaches the predetermined threshold level.

According to some embodiments, the element may include an electric circuit configured to close/open, when the liquid accumulated in the lumen is at the predetermined threshold level.

According to some embodiments, there is provided a breath sampling system including a breath sampling tube and a breath monitoring system. The breath monitoring system may monitor one or more parameters of breath samples received through the breath sampling tube. The breath sampling tube includes a filter section having a filter located within the lumen of the filter section. The filter may partially or completely block materials other than gases from passing through the breath sampling tube. The breath sampling system also includes an element fluidly coupled to the filter. The element may provide an indication, or alert, to a user that the liquid accumulated in the lumen is at or has previously reached a predetermined threshold level. In certain embodiments, the element may include an electric circuit that may close/open when the liquid accumulated in the lumen is at or has reached the predetermined threshold level. In one embodiment, the operation of the breath monitoring system may be controlled by the opening/closing of the electrical circuit.

Certain embodiments of the present disclosure may include some, all, or none of the above advantages. One or more technical effects of the present disclosure may be readily apparent to those skilled in the art from the figures, descriptions and claims included herein.

In addition to the exemplary aspects and embodiments described above, further aspects and embodiments will become apparent by reference to the figures and by study of the following detailed descriptions.

### BRIEF DESCRIPTION OF THE FIGURES

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 is a schematic diagram of a side view of a breath sampling line, in accordance with an embodiment of the present disclosure;

FIG. 2 is a schematic diagram of a filter section, in accordance with an embodiment of the present disclosure;

FIG. 3A is a schematic diagram of a filter section including a water-absorbing polymer in an active configuration, in accordance with an embodiment of the present disclosure;

FIG. 3B is a schematic diagram of a filter section including a water-absorbing polymer in an inactive configuration, in accordance with an embodiment of the present disclosure;

FIG. 4A is a schematic diagram of a filter section including a substance that may change its texture, in an active configuration, in accordance with an embodiment of the present disclosure;

FIG. 4B is a schematic diagram of a filter section including a substance that may change its texture, in an inactive configuration, in accordance with an embodiment of the present disclosure.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a fluid filtering device (e.g., filter-containing disposable) for separating liquids from gases (e.g., CO₂) to be analyzed by a monitoring system, the fluid filtering device includes a wall forming a lumen, an inlet at a proximal end of the lumen, an outlet at a distal end of the lumen, and a filter located within the lumen between the proximal and distal ends. The filter may partially or completely block materials other than gases from passing through the lumen. The fluid filtering device may include a element fluidly coupled to the filter. The element may provide an indication, or alert, to a user (e.g., a patient or caregiver) when liquid accumulated in the lumen/filter is at or has reached a predetermined threshold level.

As used herein, the term “fluid filtering device” denotes any device including a filter that may separate liquids from gases and/or block liquids from flowing through the filter, while allowing essentially unhindered and undisrupted flow of gases through the filter. The fluid filtering device may be a stand-alone element/device and/or a part of or a section of a tube, such as, but not limited to, a breath sampling tube.

As used herein, the term “element” denotes any element that may provide an irreversible indication that the filter has reached its full capacity of liquid and should be discarded. According to some embodiments, the indication may be a visual/audible or other indication directed to a user (e.g., a patient, a caregiver, or other). Additionally or alternatively, the indication may be a proactive indication, i.e., actively preventing re-use of the filter, with or without an in-built indication (e.g., a visual indication) alerting the user about the condition of the filter.

According to some embodiments, the element may change a visual property of the fluid filtering device (e.g., the filtering element) once the liquid in a lumen of the fluid filtering device or the filter reaches the predetermined threshold level. As a non-limiting example, the fluid filtering device may include a substance that may irreversibly change its color or other visual property as a result of being exposed to liquids. According to some embodiments, the indicative substance may be visible, for example, through a window formed in a wall of the fluid filtering device. According to some embodiments, the indicative substance may be disposed within and/or included in the material forming the wall of the fluid filtering device, in such manner that changes in its visible property are readily detectable.

According to some embodiments, the element may additionally or alternatively, include an electric circuit that may close/open as a result of the liquid in the lumen of the fluid filtering device and/or the filter having reached a predetermined threshold level. According to some embodiments, the electrical circuit may activate or enable activation of the fluid filtering device (or breath sampling tube coupled to the fluid filtering device) when open or when closed. As a non-limiting example, the electrical circuit, when closed, may send a signal to a medical device (e.g., capnograph), to which the fluid filtering device (or breath sampling tube) is connected, that the fluid filtering device is suitable for use. As another non-limiting example, the electrical circuit, when closed, may send a signal to a medical device (e.g. capnograph) to which the fluid filtering device (or breath sampling tube) is connected, that the fluid filtering device is unsuitable for use, and optionally interfere and/or halt the use of the fluid filtering device, for example, by deactivating a pump drawing breath samples to the medical device.

According to some embodiments, the element may include a substance. As used herein, the term “substance” is intended to denote any matter or material that may change as a result of being exposed to liquids, and that may determine the usability of the fluid filtering device, e.g. by activating an electrical circuit included therein, as a result thereof According to some embodiments, the substance may irreversibly change its property due to interaction with the liquid. As a non-limiting example, the substance may be altered when exposed to liquids. As another non-limiting example, the substance may lose its conductivity when exposed to liquids. As another non-limiting example, the substance may become conductive when exposed to liquids. As another non-limiting example, the substance may change is size (e.g. swell) when exposed to liquids. As another non-limiting example, an acidity of the substance may change when exposed to liquids. As another non-limiting example, a texture (e.g., become hard, non-porous, or otherwise block passage of gases through the fluid filtering device) of the substance may change when exposed to liquids.

According to some embodiments, the substance may be or include a water-absorbing polymer, such as, but not limited to, sodium polyacrylate.

According to some embodiments, the change in the property of the substance may close or open the electrical circuit, and as a result affect the operation of the medical device to which the fluid filtering device (or disposable including same) is connected, as described herein.

According to some embodiments, the element may be positioned within the wall of the lumen. According to some embodiments, the element may be positioned within the lumen of the fluid filtering device. According to some embodiments, the element may be positioned within a channel formed in the lumen of the fluid filtering device.

According to some embodiments, the element may include a capillary channel that may receive fluids accumulating in the lumen. According to some embodiments, once an amount of liquid absorbed by the filter and/or accumulating in the lumen reaches a predetermined threshold level, the fluids may be drawn into the capillary channel due to pressure differences created. This may advantageously allow the element to be exposed to liquids only after the fluid in the lumen of the fluid filtering device and/or absorbed by the filter is at or has reached a predetermined threshold, such as, but not limited to, the filtering element reaching 100 percent capacity, at least 95 percent capacity, at least 90 percent capacity, at least 80 percent capacity, at least 60 percent capacity or at least 50 percent capacity. Each possibility is a separate embodiment. According to some embodiments, the flow of fluid into the capillary channel or other element may be gradual, such as, but not limited to, essentially linear to the absorption of fluid by the filter.

According to some embodiments, the substance may be contained within the capillary channel.

According to some embodiments, there is provided a breath sampling tube with a filter section having a filter located within its lumen, the filter may block material other than gases from passing therethrough. The filter section further includes an element, fluidly connected to the filter, and that may provide an indication or alert to a user once the liquid in the lumen and/or the filter is at a predetermined threshold level, as disclosed herein.

According to some embodiments, the filter section may be integral to and/or in-line with the breath sampling tube. As used herein, the term “integral to” is intended to denote a filter section being attached to the breath sampling tube, for example, by being formed in a single mold/cast, by being molded on or otherwise irreversibly connected to the breath sampling tube (e.g., unless broken), thereby resulting in a single disposable breath sampling tube having an in-line filter.

Alternatively, the filter section may be a separate element connectable, optionally irreversibly, to the breath sampling tube, for example, through a connector. In some such embodiments, the filter, when unsuitable for use, may be replaced while leaving the remaining breath sampling tube intact.

According to some embodiments, the breath sampling tube may be suitable for use with a capnograph or other carbon dioxide (CO₂) monitoring system/sensor.

According to some embodiments, there is provided a breath sampling system including a breath sampling tube, a filter section including a filter located within a lumen of the filter section and that may block materials other than gases from passing therethrough, and a breath monitoring system that may monitor one or more parameters of breath samples received through the breath sampling tube.

According to some embodiments, the filter section may include an element fluidly coupled to the filter, and that may provide an indication to a user once the liquid in the lumen and/or filter has reached a predetermined threshold level, as disclosed herein.

According to some embodiments, the breath monitoring system may be a capnograph or other device/sensor that may monitor the concentration of CO₂ in a subject's exhaled breath.

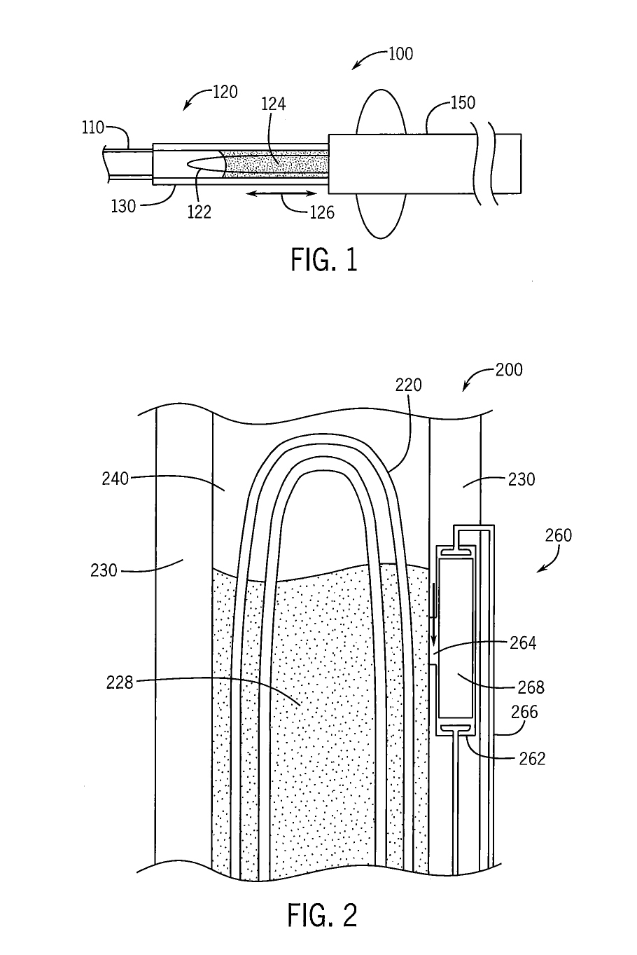

Reference is now made to FIG. 1, which illustrates an embodiment of a breath sampling line 100. Breath sampling line 100 may be connected to a breath monitoring system (not shown), such as, but not limited to, a capnograph, through connector 150 and includes a breath sampling tube 110 and a filter section 120 formed in-line with breath sampling tube 110. The filter section 120 includes a filter 122 that may separate liquids from gases, for example by absorbing and/or deflecting or otherwise reducing and/or removing moisture from a patient's exhaled breath. The filter 122 may also allow unhindered and undisrupted flow of gases through the filter 122 in a downstream direction (e.g., toward the breath monitoring system). That is, gases in the exhaled breath will be able to flow towards the breath monitoring system, while maintaining its flow characteristics, thus enabling obtaining efficient and reliable capnograms. The filter section 120 also includes a material 124 that may change a visual property when exposed to liquids. In certain embodiments, the change in the property of the material 124 may occur when the liquid in the filter 122 reaches a predetermined threshold level. Alternatively, in accordance with other embodiments, the change in the property of the material 124 may occur gradually as the amount of liquids in the filter 122 (e.g. liquid absorbed by the filter 122) increases. In the latter case, the change in the property of the material 124 may be proportional to the remaining life-time of the breath sampling line 100 and/or to the fraction of the filter 122 available for further absorption, as indicated by arrow 126. According to some embodiments, the material 124 may be visible to bystanders, for example through a window (not shown) (e.g., transparent portion) formed in a wall 130 of the filtering section 120. According to some embodiments, the material 124 may be disposed within and/or included in the material forming the wall 130 of the filtering section 120, in such a manner that changes in the visible property of the material 124 are readily detectable to bystanders.

Reference is now made to FIG. 2, which schematically illustrates a filter section 200, according to some embodiments. The filter section 200 may be part of a breath sampling line, such as, but not limited to, the breath sampling line 100. The filter section 200 includes a filter 220 that may separate liquids 228 from gases, for example by absorbing and/or deflecting or otherwise reducing and/or removing moisture from the patient's exhaled breath. The filter 220 may also allow unhindered and undisrupted flow of gases through the filter section 200 in the downstream direction (e.g., toward the breath monitoring system). The filter section 200 also includes an element 260 that may block improper reuse of the fluid filtering device 200 (and/or of the breath sampling line of which the fluid filtering device 200 is a part of). The element 260 may, as explained herein, be of various kinds and include various elements, and is characterized by its ability to mitigate improper reuse of a breath sampling line having a reconstituted filter section. The element 260 disclosed herein includes a capillary channel 262 formed in a wall 230 of the filter section 200, as shown in the illustrated embodiment. However, in other embodiments, the element 260 may be contained within the lumen 240 of the filter section 200. The element 260 is fluidly coupled to the lumen 240 of the filter section 200 through inlet 264, in such a manner that the liquids 228, absorbed by the filter 220 and/or accumulating in the lumen 240 of the filter section 200, may be drawn into the capillary channel 262, due to pressure differences created once the liquids accumulated/absorbed reach a predetermined threshold. The element 260 also includes an electrical circuit 266 including a conductive substance 268 closing electrical circuit 266. The conductive substance 268 may be sensitive to liquids. For example, the conductive substance 268 may lose its conductive property upon exposure to liquids (e.g. upon exposure to a certain amount of liquids and/or once immersed in liquids). Thus, once the liquids 228 from the lumen 240 enter the capillary channel 262, the conductive substance 268 may deteriorate, thereby opening the electrical circuit 260. Once the electrical circuit 260 is opened, the breath monitoring system to which the filter section 200 is (directly or indirectly) connected, may not receive a signal activating its operation (e.g., turning on a pump that may draw breath samples to the breath monitoring system). Further use of the filter section 200 (and/or of the breath sampling line of which it is part of) may thus be blocked. It is understood that once deteriorated, the conductive substance 268 cannot be reconstituted, and reuse of the filter section 200 is thus blocked, even if the filter 220 has been reconstituted (e.g., dried). It is further understood that the element 260 may be used alone or in conjunction with another indicator that may change a visual property thereof, when exposed to liquids, such as, but not limited to, the material 124.

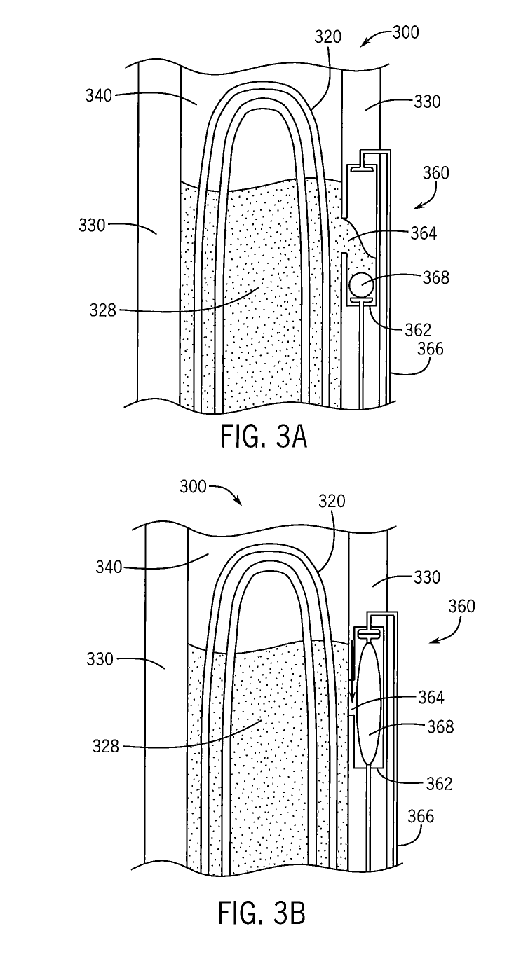

Reference is now made to FIG. 3A and FIG. 3B, illustrating a filter section 300 in an active configuration and an inactive configuration respectively, according to embodiments of the present disclosure. The filter section 300 may be part of a breath sampling line, such as but not limited to the breath sampling line 100. The filter section 300 includes a filter 320 that may separate liquids 328 from gases (e.g., CO₂), for example by absorbing and/or deflecting or otherwise reducing and/or removing moisture from the exhaled breath. The filter 320 may also allow unhindered and undisrupted flow of gases through the filter section 300 in the downstream direction toward the breath monitoring system. The filter section 300 further includes an element 360 that may block improper reuse of the fluid filtering device 300 (and/or of the breath sampling line of which it is part of). The element 360 may, as explained herein, be of various kinds and include various elements, and is characterized by its ability to block improper reuse of a breath sampling line having a reconstituted filter section. The element 360 includes a capillary channel 362 formed in a wall 330 of the filter section 300, as shown in FIGS. 3A and 3B, or contained within the lumen 340 of the filter section 300. The element 360 is fluidly coupled to the lumen 340 of the filter section 300 through an inlet 364 such that the liquids 328, absorbed by the filter 320 and/or accumulating in the lumen 340 of the filter section 300, may be drawn into the capillary channel 362. Flow of the liquids 328 into the capillary channel 362 may be due, in part, to pressure differences created once the liquids 328 accumulated/absorbed in the filter 320 reach a predetermined threshold. The element 360 further includes an electrical circuit 366 including a water-absorbing polymer 368 having a first size prior to being exposed to water. For example, in the illustrated embodiment, the water-absorbing polymer 368 is shaped in a ball sized to occupy a portion of capillary channel 362 in a non-swollen state, as illustrated in FIG. 3A. In its first size (e.g., non-swollen state), the water-absorbing polymer 368 may occupy between approximately 0.5 percent and 50 percent of a volume of the capillary channel 362 such that the water-absorbing polymer 368 does not close electrical circuit 366. As such, the breath monitoring system fluidly coupled to the filter section 300 may be unable to receive a signal interfering with its operation (e.g., turning off a pump that may draw breath samples to the breath monitoring system). The water-absorbing polymer 368 may be sensitive to liquids (e.g., the water-absorbing polymer 368) may swell when exposed to liquids. Thus, once the liquids 328 from the lumen 340 flow into the capillary channel 362, the water-absorbing polymer 368 may swell and increase in size to a second size. Upon swelling, the water-absorbing polymer 368 occupies a larger volume of the capillary channel 362 compared to when the water-absorbing polymer 368 is non-swollen, thereby closing the electrical circuit 366, as illustrated in FIG. 3B. Once the electrical circuit 366 is closed, the breath monitoring system fluidly coupled to the filter section 300 may receive a signal deactivating its operation (e.g., turning off a pump that may draw breath samples to the breath monitoring system) and further use of the filter section 300 (and/or of the breath sampling line of which it is part of) may be blocked. Once the water-absorbing polymer 368 swells due to absorption of moisture, the water-absorbing polymer 368 is unable to return to its first size (e.g., non-swollen state) and reuse of the filter section 300 is thus blocked, even if the filter 320 is reconstituted (e.g., dried). The element 360 may be used alone or in conjunction with an indicator that may change a visual property thereof, when exposed to liquids, such as but not limited to, the material 124.

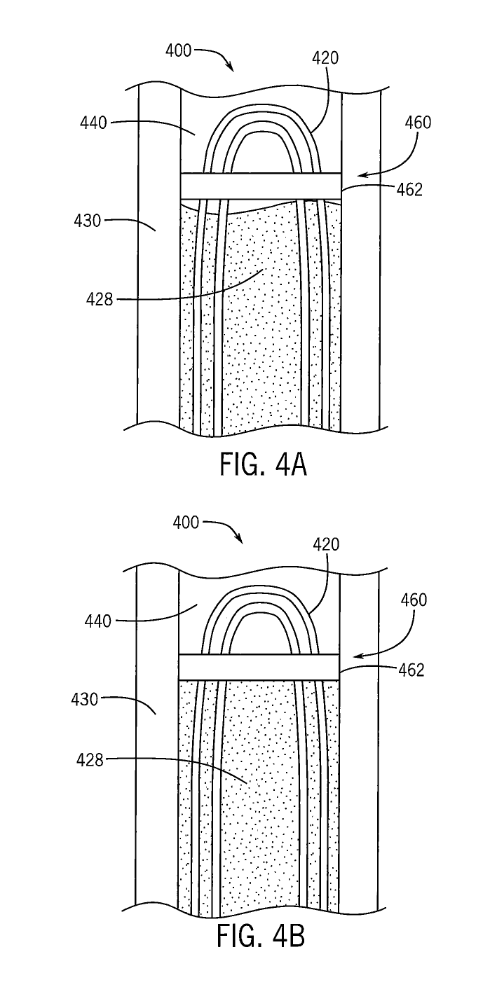

Reference is now made to FIG. 4A and FIG. 4B illustrating a filter section 400 in an active configuration and an inactive configuration respectively, according to some embodiments. The filter section 400 may be part of a breath sampling line, such as, but not limited to, the breath sampling line 100. The filter section 400 includes a filter 420 that may separate liquids 428 from gases (e.g., CO₂), for example by absorbing and/or deflecting or otherwise reducing and/or removing moisture from the exhaled breath. The filter 420 may also allow unhindered and undisrupted flow of gases through/the filter section 400 in the downstream direction toward the breath monitoring system. The filter section 400 further includes an element 460 that may block improper reuse of the fluid filtering device 400 (and/or of the breath sampling line of which it is part of). The element 460 may, as disclosed herein, be of various kinds and include various elements, and is characterized by its ability to block improper reuse of a breath sampling line having a reconstituted filter section. The element 460 in the illustrated embodiment includes a substance 462 positioned such that liquids 428, absorbed by the filter 420 and/or accumulating in the lumen 440 of the filter section 400, reach and/or come in contact with the substance 460 when a volume of the liquids 428 reach a certain level. Prior to being exposed to water, as illustrated in FIG. 4A, the substance 462 allows unhindered and undisrupted flow of gases through the filter section 400 in the downstream direction towards the breath monitoring system to which the filter section 400 is fluidly coupled to. However, upon being exposed to liquids, a texture of the substance 462 may change, as illustrated in FIG. 4B, thereby blocking the flow of the gas to the breath monitoring system, and thus further use of the filter section 400 (and/or of the breath sampling line of which it is a part of). Upon changing its texture, the substance 462 cannot return to its initial texture, and reuse of the filter section 400 is thus blocked, even if the filter 420 is reconstituted (e.g., dried). The element 460 may be used alone or in conjunction with another indicator that may change a visual property thereof, when exposed to liquids, such as, but not limited to, the material 124.

In certain embodiments, the breath monitoring system may activate an alarm in response to the filter being at or having reached the predetermined threshold level of fluids (e.g., liquid) absorbed by the filter. For example, the breath monitoring system may monitor a flow of fluid through the filter. If the flow of fluid changes (e.g., decreases), the breath monitoring system may activate an alarm to alert a caregiver that the filter is at or has reached the predetermined threshold level of fluids absorbed by the filter. In certain embodiments, the breath monitoring system may measure a moisture content of the filter. The breath monitoring system may activate an alarm when the moisture content within the filter is at or above a threshold, thereby alerting the caregiver to change the filter. In other embodiment, the breath monitoring system may receive an alarm activation signal from the filter. For example, when the electrical circuit is open or closed after the predetermined threshold level of the fluids absorbed by the filter has been reached, the breath monitoring system may activate the alarm.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof. According to some embodiments, the term “comprising” may be replaced by the term “consisting essentially of” or “consisting of”.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A fluid filtering device configured to separate liquid from gases to be analyzed, the filtering device comprising:
a wall forming a lumen, the lumen comprising an inlet at a proximal end, an outlet at a distal end, and a filter located within the lumen between the proximal end and the distal end, wherein the filter is configured to block material other than gases from passing therethrough; and
an element fluidly coupled to the filter, wherein the element is configured to provide an indication to a user that the liquid in the lumen is at a predetermined threshold level.

2. The fluid filtering device of claim 1, wherein the element is configured to change a visual property of the fluid filtering device once the liquid in the lumen is at the predetermined threshold level.

3. The fluid filtering device of claim 1, wherein the element comprises an electric circuit configured to close or open when the liquid in the lumen is at the predetermined threshold level.

4. The fluid filtering device of claim 1, wherein the element comprises a substance that is configured to irreversibly change its property due to interaction with the liquid.

5. The fluid filtering device of claim 4, wherein the substance is configured to be altered by the liquid.

6. The fluid filtering device of claim 5, wherein the substance is a conductive substance, and wherein alteration of the substance closes the electrical circuit.

7. The fluid filtering device of claim 4, wherein the substance is configured to swell upon interaction with the fluid.

8. The fluid filtering device of any of claims 4, wherein said substance comprises sodium polyacrylate.

9. The fluid filtering device of claim 7, wherein swelling of the substance closes an electrical circuit.

10. The fluid filtering device of claim 4, wherein said substance is held within said capillary channel.

11. The fluid filtering device of claim 1, wherein the element is positioned within the wall of the lumen.

12. The fluid filtering device of claim 1, wherein the element comprises a capillary channel configured to receive a portion of the liquid when the liquid in the lumen reaches the predetermined threshold level.

13. A breath sampling tube comprising:
a filter section comprising a filter located within a lumen of the filter section, wherein the filter is configured to block materials other than gases from passing therethrough; and
an element fluidly coupled to the filter, wherein the element is configured to provide an indication to a user that the liquid in the lumen is at a predetermined threshold level.

14. The breath sampling tube of claim 13, wherein the element is configured to change a visual property once the liquid in the lumen is at the predetermined threshold level.

15. The breath sampling tube of claim 13, wherein the element comprises an electric circuit configured to close and/or open when the liquid in the lumen is at the predetermined threshold level.

16. The breath sampling tube of claim 13, wherein the filter section is integral with the breath sampling tube.

17. The breath sampling tube of claim 13, wherein the filter section is a separate element from the breath sampling tube and is connectable to the breath sampling tube.

18. A breath sampling system comprising:
a breath sampling tube comprising:
a filter section comprising a filter located within a lumen of the filter section, wherein the filter is configured to block materials other than gases from passing therethrough;
an element fluidly connected to the filter, wherein the element is configured to provide an indication to a user that the liquid in the lumen is at a predetermined threshold level; and
a breath monitoring system configured to monitor one or more parameters of breath samples received through the breath sampling tube.

19. The breath sampling system of claim 18, wherein the element comprises an electric circuit configured to close or/open when the liquid in the lumen is at the predetermined threshold level.

20. The breath sampling system of claim 19, wherein an operation of the breath monitoring system is controlled by the opening or/closing of the electrical circuit.
